(19) United States
(12) Patent Application Publication
(10) Pub. No.: US 2010/0113917 A1
(43) Pub. Date: May 6, 2010

(54) SYSTEM AND METHOD FOR TRACKING OBJECT

(75) Inventor: Peter Traneus Anderson, Andover, MA (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/262,238

(22) Filed: Oct. 31, 2008

(51) Int. Cl. A61B 1/00; A61B 5/05

(52) U.S. Cl. 600/424; 600/117

(57) ABSTRACT

In one embodiment, a position transponder for operation inside the body of a subject is provided. The transponder comprises a variable resistor and a magneto resistor coupled to the variable resistor. The variable resistor comprises an electronic device having a gate terminal, a source terminal and a drain terminal and a sensor coil coupled to the electronic device between the gate terminal and the source terminal.

### FIELD OF INVENTION

The invention generally relates to intrabody tracking systems and more particularly to methods and devices for tracking the position and orientation of an object in the body.

### BACKGROUND OF THE INVENTION

Many surgical, diagnostic, therapeutic and prophylactic medical procedures require the placement of objects such as sensors, treatment units, tubes, catheters, implants and other objects within the body.

In many instances, insertion of the object is for a limited time, such as during surgery or catheterization. In other cases, objects such as feeding tubes or orthopedic implants are inserted for long-term use. A need exists for providing real-time information for accurately determining the location and orientation of objects within a patient's body, while minimizing the use of X-ray imaging.

It is known in the art to use microcoils as magnetic field transmitters and as magnetic field receivers. Further, the use of magnetic field sensors in determining the location and orientation of an object inside the patient's body is well known. Typically, the magnetic field sensor is located at the tip of a guidewire or a catheter and a plurality of leads connect the magnetic field sensor to an outside processing circuitry. The size of the magnetic field sensor located at the tip of the guidewire or the catheter is desired to be small and the number of leads connecting the sensor to the outside processing circuitry is desired to be less.

Generally, a tracking system adapted for determining the location and orientation of an object, employs at least one magnetic field sensor, the at least one magnetic field sensor comprising a plurality of coils. A first coil provides five degrees of freedom (five location and orientation coordinates) and a second coil provides the sixth degree of freedom, at the price of twice as many leads and twice as much space.

One of the prior art methods provides a magnetic field sensor using three co-located flux-gate magnetometers. A major disadvantage associated with this method is, the magnetic field sensor becomes bulky and employs a large number of leads thereby consuming more space and resource.

A number of other methods suggested in the prior art use three co-located coils and/or two non-coaxial coils (which may be co-located or positioned in Hazeltine configuration). This again is associated with a common disadvantage of using more space and resource.

Thus, there also exists a need for reducing the size of the magnetic field sensor used in tracking, as well as, the number of leads used in the tracking system.

### BRIEF DESCRIPTION OF THE INVENTION

The above-mentioned shortcomings, disadvantages and problems are addressed herein which will be understood by reading and understanding the following specification.

In one embodiment, a position transponder for operation inside the body of a subject is provided. The transponder comprises a variable resistor and a magneto resistor coupled to the variable resistor. The variable resistor comprises an electronic device having a gate terminal, a source terminal and a drain terminal and a sensor coil coupled to the electronic device between the gate terminal and the source terminal. The sensor coil is coupled such that a voltage drop is induced in the sensor coil responsive to one or more electromagnetic fields applied to the body in a vicinity of the transponder. The voltage drop across the sensor coil when applied between the gate terminal and the source terminal of the electronic device induces a voltage drop between the source terminal and the drain terminal of the electronic device. The voltage drop between the source terminal and the drain terminal of the electronic device indicates the voltage drop across the two terminals of the variable resistor The magneto resistor is coupled to the variable resistor in series, such that a voltage drop is induced in the magneto resistor responsive to the electromagnetic fields applied to the body. The transponder further comprises a control unit coupled to the variable resistor and the magneto resistor. The control unit is configured to generate an output signal indicative of the voltage drop induced at the variable resistor and the voltage drop induced at the magneto resistor, such that the output signal is indicative of coordinates of the transponder inside the body. The control unit is further configured to transmit the output signal to a signal processing unit positioned outside the body for use in determining the coordinates.

In another embodiment, a tracking system for tracking an object is provided. The tracking system comprises a radio frequency driver, adapted to transmit a radiofrequency driving current to the object, a plurality of transmitters adapted to generate electromagnetic fields at different respective frequencies, in a vicinity of the object, a transponder coupled to the object and a signal processing unit coupled to the transponder. The transponder comprises a variable resistor, a magneto resistor coupled to the variable resistor and a control unit coupled to the variable resistor and the magneto resistor. The variable resistor comprises an electronic device having a gate terminal, a source terminal and a drain terminal and a sensor coil coupled to the electronic device between the gate terminal and the source terminal. The sensor coil is configured to sense a voltage drop in response to exposure to the electromagnetic fields. The voltage drop across the sensor coil when applied between the gate terminal and the source terminal of the electronic device induces a voltage drop between the source terminal and the drain terminal of the electronic device. The voltage drop between the source terminal and the drain terminal of the electronic device indicates the voltage drop across the two terminals of the variable resistor The magneto resistor is coupled to the variable resistor in series, such that the magneto resistor is adapted to sense the electromagnetic field at a direction substantially perpendicular to the axis of the sensor coil and thereby experience a voltage drop. The control unit coupled to the variable resistor and the magneto resistor is configured to generate and transmit an output signal indicative of the voltage drop induced at the variable resistor and the voltage drop induced at the magneto resistor. Further, the signal processing unit coupled to the transponder is adapted to receive the output signal transmitted by the control unit and responsive thereto to determine the coordinates of the object.

In yet another embodiment, a tracking system for tracking an object is provided. The tracking system comprises a radio frequency driver, adapted to transmit a radiofrequency driving current to the object, a plurality of transmitters adapted to generate electromagnetic fields at different respective frequencies, in a vicinity of the object, a transponder coupled to the object and a signal processing unit coupled to the transponder. The transponder comprises a variable resistor, a magneto resistor coupled to the variable resistor and a control unit coupled to the variable resistor and the magneto resistor. The variable resistor comprises a field effect transistor having a gate terminal, a source terminal and a drain terminal and a sensor coil coupled to the field effect transistor between the gate terminal and the source terminal. The sensor coil is configured to sense a voltage drop in response to exposure to the electromagnetic fields. The voltage drop across the sensor coil when applied between the gate terminal and the source terminal of the field effect transistor induces a voltage drop between the source terminal and the drain terminal of the field effect transistor. The voltage drop between the source terminal and the drain terminal of the field effect transistor indicates the voltage drop across the two terminals of the variable resistor The magneto resistor is coupled to the variable resistor in series, such that the magneto resistor is adapted to sense the electromagnetic field at a direction substantially perpendicular to the axis of the sensor coil and thereby experience a voltage drop. The control unit coupled to the variable resistor and the magneto resistor is configured to generate and transmit an output signal indicative of the voltage drop induced at the variable resistor and the voltage drop induced at the magneto resistor. Further, the signal processing unit-coupled to the transponder is adapted to receive the output signal transmitted by the control unit and responsive thereto to determine the coordinates of the object.

In yet another embodiment, a method for tracking an object is provided. The method comprises positioning a radio frequency (RF) driver to transmit an RF driving current to the object, coupling to the object a transponder comprising a variable resistor and a magneto resistor coupled to the variable resistor, the variable resistor comprising an electronic device and a sensor coil coupled to the electronic device, driving a plurality of transmitters to generate electromagnetic fields at respective frequencies in a vicinity of the object that induce a voltage drop across the variable resistor and the magneto resistor, generating an output signal at the transponder indicative of the voltage drop across the variable resistor and the voltage drop across the magneto resistor, transmitting the output signal from the transponder and receiving and processing the output signal at a signal processing unit to determine coordinates of the object.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a block diagram of a transponder employed in a tracking system, in one embodiment;

FIG. 2 shows a schematic diagram of the transponder shown at FIG. 1;

FIG. 3 shows a block diagram of an intra-operative tracking system, in another embodiment;

FIG. 4 shows a schematic diagram of the intra-operative tracking system of FIG. 2 used in conjunction with an imaging system, in yet another embodiment; and

FIG. 5 shows a flow diagram depicting a method of tracking an object, using the tracking system of FIG. 3.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

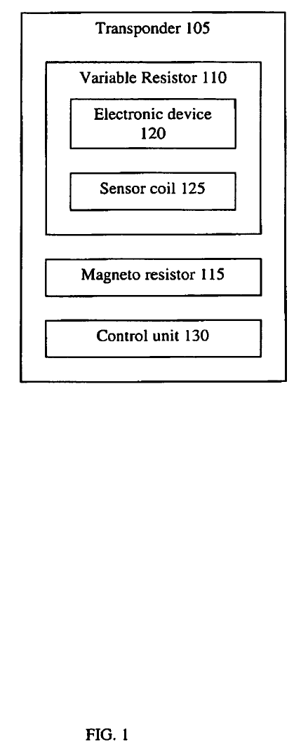

In one embodiment, shown in FIG. 1, a position transponder 105 for operation inside the body of a subject is provided. The transponder 105 comprises at least one variable resistor 110 and at least one magneto resistor 115 coupled to the variable resistor 110. The variable resistor 110 comprises an electronic device 120 and a sensor coil 125 coupled to the electronic device 120. The magneto resistor 115 is coupled to the variable resistor 110 in series such that the axis of the magneto resistor 115 is angled substantially perpendicular to the axis of the sensor coil 125. One or more electromagnetic fields are applied to the body in a vicinity of the transponder 105. The application of electromagnetic fields induce a voltage drop in each of the sensor coil 125 and the magneto resistor 115.

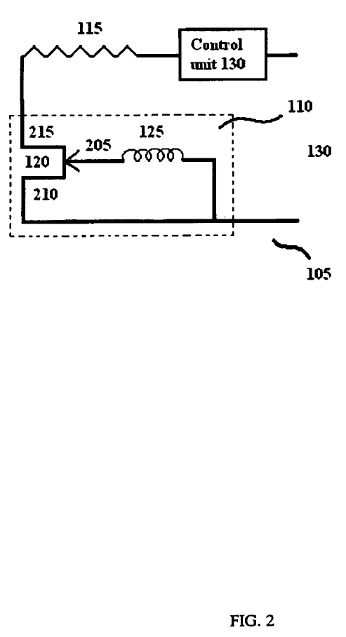

A schematic diagram of the transponder 105 is shown at FIG. 2. As shown in FIG. 2, the electronic device 120 comprises a gate terminal 205, a source terminal 210 and a drain terminal 215. In one embodiment, the electronic device 120 may comprise a, filed effect transistor (FET). The field effect transistor 120 generally implies a depletion-mode field-effect transistor (FET) that includes one of a junction FET and a Metal Oxide Semi Conductor FET (MOSFET). The field-effect transistor 120 controls the current between the source terminal 210 and drain terminal 215 by the voltage applied between the gate terminal 205 and the source terminal 210. In the field effect transistor 120, a junction between the gate terminal 205 and the source terminal 210 is generally reverse biased for control of the current between the source terminal 210 and the drain terminal 215. Generally, the field effect transistor 120 is in ON status. The application of a reverse biasing voltage causes the depletion region of that junction to expand, thereby pinching off the channel between source terminal 210 and the drain terminal 215 through which the controlled current travels. An example of the FET 120, is the 2N5457 manufactured by Fairchild Semicondutor.

As shown in FIG. 2, the sensor coil 125 is coupled to the electronic device 120 between the gate terminal 205 and the source terminal 210. Therefore, the voltage drop induced at the sensor coil 125 is applied between the gate terminal 205 and the source terminal 210 of the electronic device 120. The application of voltage between the gate terminal 205 and the source terminal 210 of the electronic device 120 controls the resistance between the source terminal 210 and the drain terminal 215 of the electronic device 120. The resistance influences the current flow between the source terminal 210 and the drain terminal 215 of the electronic device 120 thereby directly controlling the voltage drop across the source terminal 210 and the drain terminal 215 of the electronic device 120. The voltage drop between the source terminal 210 and the drain terminal 215 of the electronic device 120 indicates the voltage drop across the two terminals of the variable resistor 110.

As shown in FIG. 1, the transponder 105 further comprises a control unit 130, coupled to the variable resistor 110 and the magneto resistor 115, so as to generate an output signal indicative of the voltage drop induced at the variable resistor 110 and the voltage drop induced at the magneto resistor 115. The output signal is indicative of coordinates of the transponder 105 inside the body. The control unit 130 is further configured to transmit the output signal to a signal processing unit positioned outside the body, such that the output signal is received by the signal processing unit for use in determining the coordinates of the transponder 105.

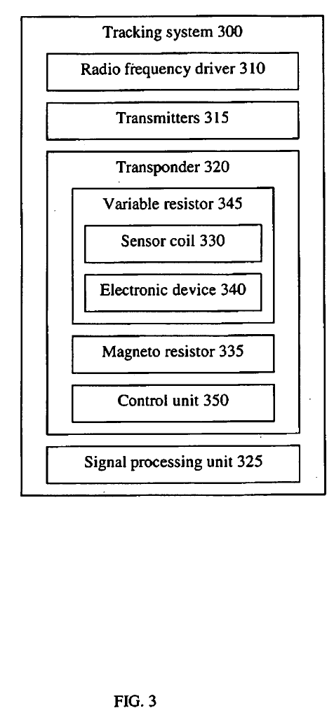

In practice, the transponder 105 is tracked against a plurality of transmitters. The plurality of transmitters emit at different respective frequencies. Further, a radiofrequency driver is configured to drive the transponder 105 with a sine wave at a selected frequency. This is further explained in conjunction with FIG. 3.

Accordingly, in one embodiment, as shown in FIG. 3, a tracking system 300 for tracking an object (not shown) is provided. The tracking system 300 comprises a radio frequency driver 310, adapted to transmit a radiofrequency driving current to the object (not shown), a plurality of transmitters 315 adapted to generate electromagnetic fields at different respective frequencies in a vicinity of the object (not shown), a transponder 320 coupled to the object (not shown) and a signal processing unit 325 coupled to the transponder 320.

The transponder 320 is typically about 2-5 mm in length and about 2-3 mm in outer diameter, enabling it to fit conveniently inside the object (not shown). The plurality of transmitters 315 emit the electromagnetic field, in the range of 2-10 kHz. The sensor coil 330 is optimized to receive and transmit high-frequency signals, in the range of 1 MHz. However, the sensor coil 330 is designed for operation in the range of 1-3 kHz, the frequencies at which the transmitters 315 generate the electromagnetic fields. Alternatively, other frequency ranges may be used, as dictated by application requirements.

Further, the sensor coil 330 has an inner diameter, of about 0.5 mm and has approximately 800 turns of about 16 micrometer diameter to provide an overall diameter in the range of 1-1.2 mm. Skilled artisans shall however appreciate that these dimensions may vary over a considerable range and are only representative of a range of dimensions. The effective capture area of the sensor coil 330 is about 400 mm·sup·2. The effective capture area is desired be made as large as feasible, consistent with the overall size requirements. Though the shape of the sensor coil 330 used in one embodiment is cylindrical, other shapes can also be used depending on the geometry of the object (not shown). An example of the sensor coil 330, is the T30AA01 passive telecoil manufactured by the Sonion division of Pulse Engineering.

With the movement of the object (not shown), the transponder 320 coupled to the object (not shown) is exposed to varying electromagnetic fields. Changing magnetic fields induce a voltage drop in the sensor coil 330. The voltage components are proportional to the strengths of the components of the respective magnetic fields produced by the transmitters 315 in a direction parallel to the axis of the sensor coil 330. The voltage drop developed at the sensor coil 330 is applied between the gate terminal 205 and the source terminal 210 of the FET 340. The current between the source terminal 210 and the drain terminal 215 of the FET 340 is controlled by the voltage applied between the gate terminal 205 and source terminal 210, thereby changing the resistance between the source terminal 210 and the drain terminal 215 of the FET 340. Thus, the variable resistor 345 comprising the sensor coil 330-and-FET 340 combination is a variable (change-of-magneto) resistor 345, where the two resistor leads are the drain terminal 215 and the source terminal 210 of the FET 340. Thus, the FET 340 along with the sensor coil 330 forms a voltage-to-resistance converter. Skilled artisans shall however appreciate that other suitable integrated circuits can be employed in place of FET 340.

The magneto resistor 335 is coupled to the variable resistor 345 in series using one of a single twisted-pair and a coaxial cable. The magneto resistor 335 is sensitive to the electromagnetic field such that the magneto resistor 335 is adapted to sense the electromagnetic field at a direction substantially perpendicular to the axis of the sensor coil 330. This configuration is aimed at minimizing the field coupling between the sensor coil 330 and the magneto resistor 335.

An example of the magneto resistor 335 is an extraordinary magneto resistance (EMR) device. Extraordinary magneto resistance (EMR) devices have been fabricated and characterized at various magnetic fields, operating temperatures, and current excitations. The extraordinary magneto resistance devices are comprised of nonmagnetic high mobility semiconductors and low resistance metallic contacts and shunts. The resistance of the extraordinary magneto resistance device is modulated by magnetic fields due to the Lorentz force steering an electron current between a high resistance semiconductor and a low resistance metallic shunt.

In order to record a significant change in the resistance of the magneto resistor 335, it is desired to drive the variable resistor 345 circuit with a current substantially below the limiting current of the FET 340, so that the FET 340 functions as a voltage-controlled resistor. However, this makes the gain of the FET 340 low.

The resistance of the variable resistor 345 and the magneto resistor 335 combination varies with the magnetic field applied to the magneto resistor 335 in addition to the change of the magnetic field applied to the sensor coil 330. For known time dependence of the magnetic field, the voltage drop across the variable resistor 345 and the voltage drop across the magneto resistor 335 can be distinguished mathematically. For example, when the electromagnetic field is a sinusoidal wave of selected frequency the resistance of the magneto resistor 335 changes sinusoidally and the resistance of the variable resistor 345 changes consinusoidally. Following ohm's law V=IR, the voltage drop across the variable resistor 345 and the voltage drop across the magneto resistor 335 are directly proportional to the resistance of the variable resistor 345 and the resistance of the magneto resistor 335 respectively. Thus, the variable resistor 345 and the magneto resistor 335 can be configured to act as two sensors with distinguishable signals connected in series across a single pair of leads.

For a sinusoidal electromagnetic field, the variation in the resistance of the magneto resistor 335 is in phase with the electromagnetic field. However, the variation in the resistance of the variable resistor 345 is out of phase with the electromagnetic field by approximately ninety degrees. Thus the two signals can be distinguished by the difference in the phases of the respective voltage drops.

The control unit 350 coupled to the variable resistor 345 and the magneto resistor 335 comprises suitable circuitry for reading the signals from the variable resistor 345 and the magneto resistor 335. For example, in one embodiment, the control unit 350 comprises at least one of a balanced bridge and hybrid-circuit electronics to read the signals, in the presence of the signals from the radio frequency driver 310. Skilled artisans shall however appreciate other suitable circuits and methods for signal processing.

Responsive to reading the signals from the variable resistor 345 and the magneto resistor 335, the control unit 350 generates an output signal indicative of an amplitude of the voltage drop induced at the variable resistor 345, an amplitude of the voltage drop induced at the magneto resistor 335, a phase of the voltage drop induced at the variable resistor 345 relative to the phase of the electromagnetic fields and a phase of the voltage drop induced at the magneto resistor 335 relative to a phase of the electromagnetic fields. The signal processing unit 325 is adapted to determine the coordinates and an orientation of the object (not shown), responsive to the amplitude and the phase of the voltage drop indicated by the output signal. Skilled artisans shall however appreciate that both analog and digital embodiments of signal processing are possible.

The signal processing unit 325 represents an assemblage of units to perform intended functions. For example, such units may receive information or signals, process information, function as a controller, display information, and/or generate information or signals. Typically the signal processing unit 325 may comprise one or more microprocessors.

Thus, the transponder 320, as described above, can be employed to provide all six position and orientation coordinates (X, Y, Z, yaw, pitch and roll) of the object (not shown). The single magneto resistor 335 shown in FIG. 3, in conjunction with one or more transmitters 315, enables the signal processing unit 325 to generate three dimensions of position and two dimensions of orientation information. The third dimension of orientation (typically rotation of the object (not shown) about its longitudinal axis) can be inferred from the variable resistor 345.

When operating at low frequencies, the sensor coil 330 is less sensitive than the magneto resistor 335. Thus the magneto resistor 335 can be employed as a first receiver providing five degree of freedom (“5DOF”) location information and consequently the variable resistor 345 can be used as a second receiver employed to track roll when operating at higher frequencies. Accordingly, it is desirable to assign the highest frequencies to the transmitters 315 useful for providing roll determination. For example, the three highest frequencies can be assigned to three transmitters 315 providing relatively uniform fields in the X, Y, and Z directions.

The voltage drop at the sensor coil 330 is small and so is the voltage between the gate terminal 205 and the source terminal 210 of the FET 340. Assuming the conductance (1/resistance) is linear, the change of resistance in the variable resistor 345 is small. Thus, the signal representing the voltage drop at the variable resistor 345 is small, however, sufficient for providing the roll information. Since the position, azimuth, and elevation are determined by the signal from the magneto resistor 335, the noise in the signal from the variable resistor 345 is present only in determining the roll information.

Thus, the magneto resistor 335, which is comparatively more sensitive than the variable resistor 345 can be used as a five degree of freedom (“5DOF”) electromagnetic tracker sensor. Subsequently, the variable resistor 345 can be employed to provide the sixth degree of freedom or to track roll.

In an alternative embodiment, the variable resistor 345 can be employed to provide five degree of freedom (“5DOF”) location information and subsequently the magneto resistor 335 can be employed to provide the roll information.

The description above primarily concerns acquiring information by a combination of a variable resistor 345 and a magneto resistor 335, used in determining the position and orientation of a remote object (not shown) such as a medical device or instrument. It is also within the scope of the invention that the transponder 320 may comprise more than one set of variable resistors or magneto resistors that provide sufficient parameters to determine the configuration of the remote object (not shown), relative to a reference frame.

Accordingly, in one embodiment, one or more magneto resistors can be combined with one or more variable resistors to obtain six position and orientation coordinates for the object (not shown). For example, a plurality of magneto resistors can be used along with one or more variable resistors or a plurality of variable resistors can be used along with one or more magneto resistors to form a transponder 320. Further, each magneto resistor can be connected to a single variable resistor using a single pair of leads.

In an alternative embodiment, the transponder 320 can be tracked against a plurality of receivers. Accordingly, the tracking system 300 can comprise a plurality of receivers and the magneto resistor 335 can be selected to be a five degree of freedom (“5DOF”) transmitter. Further, similar to the tracking system 300 described above, the variable resistor 345 can be employed to provide the roll information

In yet another alternative embodiment, the transponder 320 can be tracked against an array comprising at least one transmitter and at least one receiver. Further, each receiver can comprise a magnetic field sensor such as but not limited to a variable resistor 345.

The tracking system 300 described in various embodiments can be used as a part of a surgical navigation product. For this application, the transponder 320 is adapted to be inserted, together with the object (not shown), into the body of the subject, while one or more transmitters 315 and the RF driver 310 are placed outside the body.

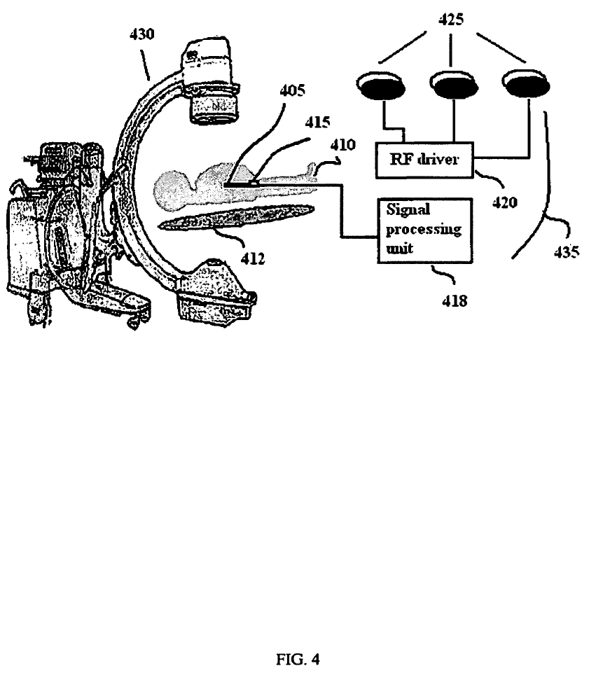

In an exemplary embodiment, shown at FIG. 4, an object 405 includes an elongate probe, for insertion into the body of a subject 410 positioned on a patient positioning system 412. A transponder 415 is fixed to the probe so as to enable an externally located signal processing unit 418 to determine the coordinates of a distal end of the probe. Alternatively, the object 405 includes an implant, and the transponder 415 is fixed in the implant so as to enable the signal processing unit 418 to determine the coordinates of the implant within the body. Further, the transponder 415 may be fixed to other types of invasive tools, such as endoscopes, catheters and feeding tubes, as well as to other implantable devices, such as orthopedic implants.

An externally-located radio frequency driver 420 sends a radio frequency (RF) signal, having a frequency in the kilohertz range, to drive the transponder 415. Additionally, a plurality of electromagnetic transmitters 425 positioned in fixed locations outside the body produce electromagnetic fields at different, respective frequencies, typically in the kilohertz range. These fields induce voltage in the sensor coil 330 and the magneto resistor 335 of the transponder 415, which depend on the spatial position and orientation of the sensor coil 330 and the magneto resistor 335 relative to the transmitters 425. The voltage drop induced at the sensor coil 330 due to varying electromagnetic field is applied between the gate terminal 205 and the source terminal 210 of the FET 340. The FET 340 converts the sensor coil 330 into a variable resistor 345. In other words, the FET 340 operates as a variable resistor 345 controlled by the sensor coil 330. Since the voltage drop induced at the sensor coil 330 is dependent on the varying electromagnetic field, the resistance developed at the FET 340 is sensitive to the rate of change of the electromagnetic field. Further, the resistances developed across the variable resistor 345 and the magneto resistors 335 are directly proportional to the voltage drops induced at the variable resistor 345 and the magneto resistor 335 respectively.

The control unit 350 converts the voltages into high-frequency signals, which in the form of the output signal is transmitted by the control unit 350 to the externally-located signal processing unit 418. The signal processing unit 418 processes the output signal to determine the position and orientation coordinates of the transponder 415 for display and recording.

Typically, prior to performing a medical procedure, the image of the subject 410 is captured using an imaging device 430 (such as an X-ray imaging device) and is displayed on a computer monitor. The transponder 415 is visible in the X-ray image, and the position of the transponder 415 in the image is registered with respective location coordinates, as determined by the signal processing unit 418. During the medical procedure, the movement of the transponder 415 is tracked by the tracking system 435 and is used to update the position of the transponder 415 in the image on the computer monitor, using image processing techniques known in the art. The updated image can be used to achieve desired navigation of the object 405 during the medical procedure, without the need for repeated X-ray exposures during the medical procedure.

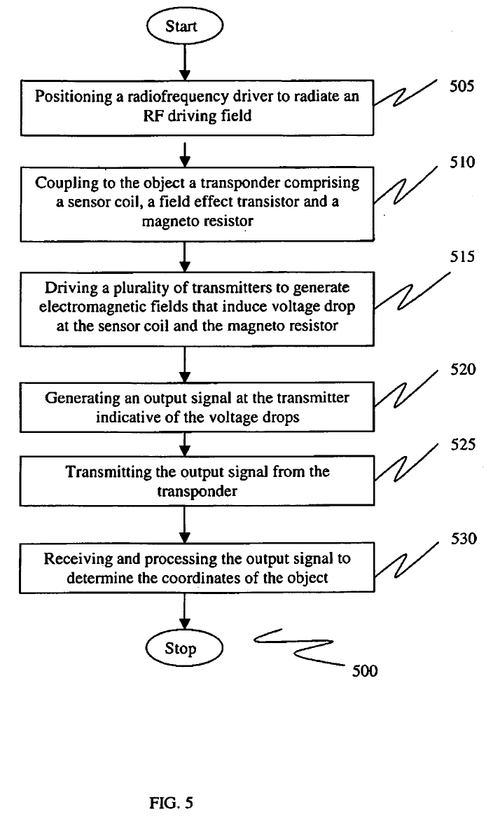

In another embodiment shown at FIG. 5, a method 500 for tracking an object 405 is provided. The method 500 comprises positioning the radio frequency (RF) driver 420 to transmit an RF driving current to the object 405 step 505, coupling to the object 405 the transponder 415 comprising the variable resistor 345 and the magneto resistor 335 coupled to the variable resistor 345 step 510, driving the plurality of transmitters 425 to generate electromagnetic fields at respective frequencies in a vicinity of the object 405 that induce a voltage drop across the variable resistor 345 and the magneto resistor 335 step 515, generating an output signal at the transponder 415 indicative of the voltage drop across the variable resistor 345 and the voltage drop across the magneto resistor 335 step 520, transmitting the output signal from the transponder 415 to the signal processing unit 418 step 525 and receiving and processing the output signal at the signal processing unit 418 to determine coordinates of the object 405 step 530.

In some embodiments, the method 500 includes inserting the transponder 415, together with the object 405, into a body of a subject 410, wherein positioning the plurality of the transmitters 425 and the RF driver 420 includes placing the one or more transmitters 425 and the RF driver 420 outside the body.

In an exemplary embodiment, to operate the transponder 415, a subject 410 is placed in a magnetic field generated, for example, by situating under the subject 410 a pad containing a plurality of transmitters 425 for generating a magnetic field. The plurality of transmitters 425 are configured to generate electromagnetic fields at different, respective frequencies. A reference electromagnetic field sensor (not shown) is fixed relative to the subject 410, for example, taped to the back of the subject 410, and the object 405 with the transponder 415 coupled thereto, is advanced into the body of the subject 410. Signals received from the transponder 415 are conveyed to the signal processing unit 418, which analyzes the signals and then displays the results on a monitor. By this method, the precise location of transponder 415, relative to the reference sensor (not shown), can be ascertained and visually displayed. Furthermore, the reference sensor (not shown) may be used to correct for breathing motion or other movement in the subject 410. In this way, the acquired position and orientation may be referenced to an organ structure and not to an absolute outside the reference frame, which is less significant.

As described in various embodiments, the invention combines a sensor coil 330 and a field effect transistor with a magneto resistor 335 to obtain a transponder 320. The magneto resistor 335 replaces a second sensor coil 330 typically employed in prior art systems, thereby eliminating the use of the second sensor coil 330. A major advantage associated with the magneto resistor 335 is its ability to be fabricated as a miniature device. Thus, replacing the second sensor coil 330 with a magneto resistor 335 smaller than the second sensor coil 330 reduces the space needed.

Further, the magneto resistor 335 and the variable resistor 345 can share a single pair of leads. This allows for a simplified guide wire fabrication as the number of leads employed in connecting two components is reduced by half. Thus, the combination of the variable resistor 345 and the magneto resistor 335 enables the transponder 320 to obtain six degrees of freedom (“6DOF”) without causing much burden on resource or space.

In various embodiments, system and method for tracking an object are described. However, the embodiments are not limited and may be implemented in connection with different applications. The application of the invention can be extended to other areas, For example, in cardiac applications such as in catheter or flexible endoscope for tracking the path of travel of the catheter tip, to facilitate laser eye surgery by tracking the eye movements, in evaluating rehabilitation progress by measuring finger movement, to align prostheses during arthroplasty procedures and further to provide a stylus input for a Personal Digital Assistant (PDA). The invention provides a broad concept of tracking an object in obscure environment, which can be adapted to track the position of items other than medical devices in a variety of applications. That is, the tracking system may be used in other settings where the position of an object in an environment is unable to be accurately determined by visual inspection. For example, tracking technology may be used in forensic or security applications. Retail stores may use tracking technology to prevent theft of merchandise. Tracking systems are also often used in virtual reality systems or simulators. Accordingly, the invention is not limited to a medical device. The design can be carried further and implemented in various forms and specifications.

This written description uses examples to describe the subject matter herein, including the best mode, and also to enable any person skilled in the art to make and use the subject matter. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A position transponder for operation inside the body of a subject, the transponder comprising:
a variable resistor comprising an electronic device and a sensor coil coupled to the electronic device such that a voltage drop is induced in the sensor coil responsive to one or more electromagnetic fields applied to the body in a vicinity of the transponder;
a magneto resistor coupled in series to the variable resistor, such that a voltage drop is induced in the magneto resistor responsive to the electromagnetic fields applied to the body; and
a control unit, coupled to the variable resistor and the magneto resistor so as to generate an output signal indicative of the voltage drop induced at the variable resistor and the voltage drop induced at the magneto resistor, such that the output signal is indicative of coordinates of the transponder inside the body.

2. The transponder of claim 1, wherein the sensor coil is coupled to the variable resistor such that the sensor coil and the magneto resistor are collocated and the axis of the magneto resistor is substantially perpendicular to the axis of the sensor coil.

3. The transponder of claim 1, wherein the electronic device includes a gate terminal, a source terminal and a drain terminal and the sensor coil is coupled to the electronic device between the gate terminal and the source terminal

4. The transponder of claim 3, wherein the electronic device is a field effect transistor (FET).

5. The transponder of claim 4, wherein the field effect transistor (FET) is one of a junction field effect transistor (JFET) and a metal oxide semi conductor field effect transistor (MOSFET).

6. The transponder of claim 1, wherein the control unit is further configured to transmit the output signal to a signal processing unit positioned outside the body for use in determining the coordinates.

7. The transponder of claim 6, wherein the control unit is adapted to generate the output signal indicative of an amplitude of the voltage drop and a phase of the voltage drop, and wherein the signal processing unit is adapted to determine the coordinates and an orientation of the object, responsive to the amplitude and the phase of the voltage drop indicated by the output signal.

8. The transponder of claim 7, wherein the control unit comprises one of a balanced bridge and hybrid circuit electronics.

9. A tracking system for tracking an object comprising:
a radio frequency driver, adapted to transmit a radiofrequency driving current to the object;
a plurality of transmitters adapted to generate electromagnetic fields at different respective frequencies in a vicinity of the object;
a transponder coupled to the object, the transponder comprising:
a variable resistor comprising an electronic device and a sensor coil coupled to the electronic device such that the sensor coil is configured to sense a voltage drop in response to exposure to the electromagnetic fields;
a magneto resistor coupled to the variable resistor in series, such that the magneto resistor and the sensor coil are co-located and the magneto resistor is adapted to sense the electromagnetic field at a direction substantially perpendicular to the axis of the sensor coil and thereby experience a voltage drop; and
a control unit coupled to the variable resistor and the magneto resistor so as to generate an output signal indicative of the voltage drop induced at the variable resistor and the voltage drop induced at the magneto resistor; and
a signal processing unit coupled to the transponder, the signal processing unit adapted to receive the output signal transmitted by the control unit and responsive thereto to determine the coordinates of the object.

10. The tracking system of claim 9, wherein the electronic device includes a gate terminal, a source terminal and a drain terminal and the sensor coil is coupled to the electronic device between the gate terminal and the source terminal

11. The tracking system of claim 10, wherein the electronic device is a field effect transistor (FET).

12. The tracking system of claim 11, wherein the field effect transistor (FET) is one of a junction field effect transistor (JFET) and a metal oxide semi conductor field effect transistor (MOSFET).

13. The tracking system of claim 9, wherein the control unit is adapted to generate the output signal indicative of an amplitude of the voltage drop and a phase of the voltage drop, and wherein the signal processing unit is adapted to determine the coordinates and an orientation of the object, responsive to the amplitude and the phase of the voltage drop indicated by the output signal.

14. The tracking system of claim 9, wherein the control unit comprises one of a balanced bridge and hybrid circuit electronics.

15. The tracking system of claim 9, wherein the output signal is analog.

16. The tracking system of claim 9, wherein the output signal is digital.

17. The tracking system of claim 9, wherein the object is a catheter or an endoscope.

18. A tracking system for tracking an object comprising:
a radio frequency driver, adapted to transmit a radiofrequency driving current to the object;
a plurality of transmitters adapted to generate electromagnetic fields at different respective frequencies in a vicinity of the object;
a transponder coupled to the object, the transponder comprising:
a variable resistor comprising a field effect transistor and a sensor coil coupled to the field effect transistor such that the sensor coil is configured to sense a voltage drop in response to exposure to the electromagnetic fields;
a magneto resistor coupled to the variable resistor in series, such that the magneto resistor and the sensor coil are co-located and the magneto resistor is adapted to sense the electromagnetic field at a direction substantially perpendicular to the axis of the sensor coil and thereby experience a voltage drop; and
a control unit coupled to the variable resistor and the magneto resistor so as to generate an output signal indicative of the voltage drop induced at the variable resistor and the voltage drop induced at the magneto resistor; and
a signal processing unit coupled to the transponder, the signal processing unit adapted to receive the output signal transmitted by the control unit and responsive thereto to determine the coordinates of the object.

19. The tracking system of claim 18, wherein the field effect transistor includes a gate terminal, a source terminal and a drain terminal and the sensor coil is coupled to the field effect transistor between the gate terminal and the source terminal.

20. The tracking system of claim 19, wherein the field effect transistor (FET) is one of a junction field effect transistor (JFET) and a metal oxide semi conductor field effect transistor (MOSFET).

21. The tracking system of claim 18, wherein the control unit is adapted to generate the output signal indicative of an amplitude of the voltage drop and a phase of the voltage drop, and wherein the signal processing unit is adapted to determine the coordinates and an orientation of the object, responsive to the amplitude and the phase of the voltage drop indicated by the output signal.

22. The tracking system of claim 18, wherein the control unit comprises one of a balanced bridge and hybrid circuit electronics.

23. The tracking system of claim 18, wherein the output signal is analog.

24. The tracking system of claim 18, wherein the output signal is digital.

25. The tracking system of claim 18, wherein the object is a catheter or an endoscope.

26. A method for tracking an object, comprising:
positioning a radio frequency (RF) driver to transmit an RF driving current to the object;
coupling to the object a transponder comprising a variable resistor and a magneto resistor coupled to the variable resistor, the variable resistor comprising an electronic device and a sensor coil coupled to the electronic device;
driving a plurality of transmitters to generate electromagnetic fields at respective frequencies in a vicinity of the object that induce a voltage drop across the variable resistor and the magneto resistor;
generating an output signal at the transponder indicative of the voltage drop across the variable resistor and the voltage drop across the magneto resistor;
transmitting the output signal from the transponder; and
receiving and processing the output signal to determine coordinates of the object.

27. The method of claim 26, further comprising inserting the transponder, together with the object, into the body of a subject.

28. The method of claim 26, wherein positioning the plurality of transmitters and the RF driver comprises placing the plurality of transmitters and the RF driver outside the body.

29. The method of claim 26, wherein the sensor coil is coupled to the variable resistor such that the sensor coil and the magneto resistor are collocated and the axis of the magneto resistor is substantially perpendicular to the axis of the sensor coil.

30. The method of claim 26, wherein the electronic device is a field effect transistor (FET).

31. The method of claim 30, wherein the field effect transistor (FET) is one of a junction field effect transistor (JFET) and a metal oxide semi conductor field effect transistor (MOSFET).
